(19) United States
(12) Patent Application Publication
(10) Pub. No.: US 2010/0116406 A1
(43) Pub. Date: May 13, 2010

(54) Wax Emulsion for Use in Building Products

(75) Inventors: Dennis Michael Mahoney, Chester Springs, PA (US); John Hartley Burns, Gap, PA (US)

Correspondence Address: FLASTER/GREENBERG P.C.;Four Penn Center, 1600 John F. Kennedy Boulevard, 2nd Floor, PHILADELPHIA, PA, 19103, US

(73) Assignee: Henry Company, El Segundo, CA (US)

(21) Appl. No.: 12/614,984

(22) Filed: Nov. 9, 2009

Related U.S. Application Data
(60) Provisional application No. 61/112,468, filed on Nov. 7, 2008.

(51) Int. Cl. C09D 191/00; C09D 129/04; C04B 24/08; C04B 24/26; B32B 37/00; C07C 11/00

(52) U.S. Cl. 156/39; 106/270; 524/557; 106/660; 524/5; 585/9

(57) ABSTRACT

Provided herein are an aqueous wax emulsion, comprising water; a paraffinic hydrocarbon; polyvinyl alcohol and a wax component comprising synthetic olefin wax component. The synthetic olefin wax component may be selected from the group consisting of (i) a synthetic normal α-olefin wax; (ii) a synthetic olefin wax of a carbon chain length of about 20 or more carbon atoms, that is modified by oxidizing and/or by refining through distillation or stripping; and (iii) combinations thereof. Such emulsions are also useful for and disclosed herein are settable gypsum compositions and water-resistant wallboard formed therefrom. Also included herein is a montan wax substitute for use in an aqueous montan-based wax emulsion having paraffinic hydrocarbon, a wax component, water and polyvinyl alcohol wherein the wax component includes the montan wax substitute in place of use of a montan wax in the wax component such that the montan wax substitute is about 1 part to about 100 percent of the wax component and includes a synthetic olefin wax.

### CROSS-REFERENCE TO RELATED APPLICATIONS

This application claims the benefit under 35 U.S.C. §119(e) of U.S. Provisional Patent Application No. 61/112,468, filed Nov. 7, 2008, the entire disclosure of which is incorporated herein by reference.

### BACKGROUND OF THE INVENTION

1. Field of the Invention

The present invention involves a wax emulsion which provides excellent moisture resistant properties for use in building materials without requiring use of montan wax.

2. Description of Related Art

Synthetic and natural waxes are used in many industries. Such wax emulsions are known for use in products within the building products industry, notably in gypsum wallboard for waterproofing and in oriented strand board. Amongst natural waxes used in the building products industry, particularly for water-resistant gypsum wallboard, montan wax is prevalent. Montan wax is a lignite-wax, including chemical components formed of long chain alkyl acids and alkyl esters having chain lengths of about 24 to 30 carbons. In addition, natural montan includes resin acids, polyterpenes and some alcohol, ketone and other hydrocarbons such that it is not a “pure” wax. The saponification number of montan, which is a saponifiable wax, is about 92 and its melting point is about 80° C. Montan wax while highly effective has its drawbacks in that it is not always sufficiently pure and as a natural wax, tends to have some inconsistencies in formulation and more importantly, is available only in limited supply from a natural source which is generated primarily in Germany, such that the wax is becoming more expensive and obtaining adequate supply is becoming an issue for manufacturers of such wax emulsions.

U.S. Pat. No. 5,437,722 describes a water-resistant gypsum composition and wax emulsion therefore, which includes a paraffin hydrocarbon having a melting point of about 40° C. to 80° C., about 1 to 200 parts by weight montan wax per 100 parts of the paraffin hydrocarbon, and about 1 to 50 parts by weight polyvinyl alcohol per 100 parts of the paraffin hydrocarbon. The use of montan wax in the wax emulsion for water-resistant wallboard has been very effective and provides excellent performance, even in view of the other drawbacks associated with use of montan wax.

In addition to montan wax, other naturally derived waxes are known for use in various industries and include petroleum waxes derived from crude oil after processing, which include macrocrystalline wax, microcrystalline wax, petrolatum and paraffin wax. Paraffin wax is also a natural wax derived from petroleum and formed principally of straight-chain alkanes having average chain lengths of 20-30 carbon atoms.

Also outside of the building products context, in addition to waxes that occur in natural form, there are various known synthetic waxes which include synthetic polyethylene wax of low molecular weight, i.e., molecular weights of less than about 10,000, and polyethylenes that have wax-like properties. Such waxes can be formed by direct polymerization of ethylene under conditions suitable to control molecular weight. Polyethylenes with molecular weights in about the 2,000-4,000 range are waxes, and when in the range of about 4,000-12,000 become wax resins.

Fischer-Tropsch waxes are polymethylene waxes produced by a particular polymerization synthesis, specifically, a Fischer-Tropsch synthesis (polymerization of carbon monoxide under high pressure, high temperature and special catalysts to produce hydrocarbon, followed by distillation to separate the products into liquid fuels and waxes). Such waxes (hydrocarbon waxes of microcrystalline, polyethylene and polymethylene types) can be chemically modified by, e.g., air oxidation (to give an acid number of 30 or less and a saponification number no lower than 25) or modified with maleic anhydride or carboxylic acid. Such modified waxes are more easily emulsified in water and can be saponified or esterified. Other known synthetic waxes are polymerized α-olefins. These are waxes formed of higher α-olefins of 20 or more carbon atoms that have wax like properties. The materials are very branched with broad molecular weight distributions and melting points ranging about 54° C. to 75° C. with molecular weights of about 2,600 to 2,800. Thus, waxes differ depending on the nature of the base material as well as the polymerization or synthesis process, and resulting chemical structure, including the use and type of any chemical modification.

In the building products area, U.S. Patent Publication No 2007/0181035 A1 is directed to a composition for use in making medium density fiberboard (MDF). The composition has a component for reducing surface tension and improving dimensional stability for use in oriented strand board and MDF. The surface tension agents are either fluorinated hydrocarbon compounds of two to six carbons or alkoxylates of alkyl phenols or alkylated acetylene diols. These materials are provided to a composition having a combination of montan wax with other waxes, ammonium hydroxide for saponification, water and polyvinyl alcohol. Nonsaponifiable waxes may be used in this composition, including paraffin and scale or slack wax (which is petroleum derived). Saponifiable waxes which may be used include Montan, petroleum wax, and various natural waxes.

U.S. Patent Publication No. 2007/0245931 A1 discloses use of alkyl phenols in emulsions for water-proof gypsum board. The alkyl phenols are long-chain hydrocarbon chains having a phenolated ring of 24-34 carbon chain lengths. The publication describes use of lignosulfonic acid, and magnesium sulfate. The wax components can be combinations of paraffin and montan. The patent claims that the compositions are stable without the use of starch as in prior U.S. Pat. No. 6,663,707 of the same inventor. The wax used in the composition may be various commercially known waxes having a melting point of from about 120° F. (48.9° C.) to 150° F. (65.6° C.) with low volatility and a high molecular weight with carbon chain lengths of 36 or higher. The hydrocarbon wax component includes waxes known in the field of gypsum slurries.

U.S. Pat. No. 6,890,976 describes an aqueous emulsion for gypsum products with hydrocarbon wax, polyolefin-maleic anhydride graft polymer and polyvinyl alcohol and/or acetate. The maleic-modified material is known as FLOZOL®. The hydrocarbon wax can be paraffin or a polyethylene wax, maleated hydrocarbon wax or combinations thereof. The wax can also be a synthetic wax ester or an acid wax. The polyolefin-maleic anhydride graft copolymer is a 50-500 carbon chain graft copolymer, which when provided to the wax emulsion is described as providing improved water repellency to a final gypsum product.

U.S. Patent Publication No. 2004/0083928 A1 describes a suspension, instead of an emulsion, of various waxes in water that is mixed directly with gypsum. In describing the waxes, the suspensions can include polyethylene wax, maleated hydrocarbons and other waxes as well as wax combinations.

U.S. Pat. No. 7,192,909 describes use of polyolefin wax in an application outside the building products area, which is as a lubricant for plastics processing, specifically for PVC. The waxes are described as homopolymers and copolymers of various α-olefins that have been modified in a polar manner (oxidized) or grated with polar reagents. They can be used alone or in combination with other waxes, e.g. montan waxes, fatty acid derivatives or paraffins.

U.S. Publication No. 2006/0196391 describes use of triglycerides in emulsions, and notes that the prior art has made use of petroleum waxes and synthetic waxes such as Fischer Tropsch and polyethylene waxes, which have been used for purposes similar to those of the invention of Publication 2006/0196391 with mixed results.

Various types of α-olefin and other olefinic synthetic waxes are known within the broad category of waxes, as are chemically modified waxes, and have been used in a variety of applications, outside the water-resistant wallboard area. They are of a wide variety and vary in content and chemical structure. As noted above, water-resistant wallboard products generally use paraffin or montan in a formulation alone or in combination with each other, or other paraffinic or synthetic waxes as described above in the mentioned exemplary patent references. While various waxes and wax substitutes have been used and tried in the building products area for wax emulsions generally, particularly in some cases with a goal toward finding an adequate substitute for use of montan wax, the waxes as have been adopted to date do not include normal α-olefin or oxidized α-olefin waxes.

There is a need in the art for continued development of wax emulsions for use in building products such as water-resistant gypsum compositions and oriented strand board, as well as a desire to find substitutes for the costly use of montan wax, which is in limited supply and can have inconsistencies due to its natural source, while still delivering the same waterproofing properties and desired wax emulsion properties of montan wax.

### BRIEF SUMMARY OF THE INVENTION

The present invention includes an aqueous wax emulsion that comprises: water, a paraffinic hydrocarbon, and a wax component comprising synthetic olefin wax component, wherein the synthetic olefin wax component is selected from the group consisting of (i) a synthetic normal α-olefin wax; (ii) a synthetic olefin wax of a carbon chain length of about 20 or more carbon atoms, that is modified by oxidizing and/or by refining through distillation or stripping; and (iii) combinations thereof. The synthetic olefin wax component may be the synthetic olefin wax (ii) having a carbon chain length of at least 28, more preferably at least 30. The synthetic olefin wax component may also be the synthetic olefin wax (ii) and comprise a mixture of one or more of (a) an olefin having a carbon chain of about 28 to about 54 carbons; (b) one or more component selected from an aldehyde, a ketone, a carboxylic acid and a carboxylic ester; and (c) a dimer prepared from olefins having carbon chain lengths of about 28 to about 54 so as to have dimer carbon chain lengths of about 65 to about 108.

In one embodiment, the wax component may further comprise montan wax in a blend with the synthetic olefin wax component, or be present in a blend of the synthetic olefin wax component with one or more of the following components: natural or synthetic carnauba wax, palm wax, Fischer-Tropsch wax, a polymeric alkene, and an oxidized polyethylene wax. In another embodiment, the synthetic olefin wax component is from about 1 percent to about 100 percent of the wax component, preferably about 20 percent to about 80 percent of the wax component, more preferably about 30 percent to about 70 percent of the wax component and most preferably about 40 percent to about 60 percent of the wax component. In further embodiments, the emulsion may comprise a saponifying agent. The saponifying agent may be an alkali metal, such as potassium hydroxide. The emulsion may also include at least one of a dispersant and a surfactant. Such dispersants preferably comprise sulfur or a sulfur-containing group, and may be, for example, lignosulfonate. In preferred embodiments herein, the paraffinic hydrocarbon is a paraffin wax having a melting point of about 40° C. to about 80° C.

In yet further embodiments, the emulsion further comprises a stabilizer, such as, for example, polyvinyl alcohol, which may be present in an amount of about 1 part to about 20 parts, by weight, per 100 parts of said paraffin hydrocarbon. In addition, the polyvinyl alcohol is preferably about 97% to about 100% hydrolyzed polyvinyl alcohol.

The invention also includes a settable gypsum composition suitable for forming a water-resistant gypsum product comprising: a) 100 parts by weight of gypsum, and b) about 0.5 part to about 20 parts, by weight, of emulsion solids, per 100 parts, by weight, of gypsum, of an aqueous emulsion comprising: i) water; ii) a paraffinic hydrocarbon; and iii) a wax component comprising synthetic olefin wax component, wherein the wax component is present in an amount of about 1 part to about 200 parts, by weight, per 100 parts of the paraffinic hydrocarbon. The synthetic olefin wax component may be as described hereinabove. In one embodiment, the emulsion further comprises polyvinyl alcohol, which may be present in an amount of about 1 part to about 50 parts, by weight, per 100 parts of said paraffin hydrocarbon. In preferred embodiments herein, the polyvinyl alcohol may be about 97% to about 100% hydrolyzed polyvinyl alcohol.

The invention further includes a water-resistant gypsum board comprising a set composition of the type of gypsum composition noted herein above. In a further embodiment, the board may have a core sandwiched between a pair of liners, wherein the core comprises a set composition of the type of gypsum composition noted herein above.

The invention further includes a method of manufacturing a water-resistant gypsum board comprising: a) forming a mixture of: i) 100 parts by weight of gypsum; and ii) about 0.5 part to about 20 parts, by weight, of emulsion solids, per 100 parts, by weight, of the gypsum, of an aqueous emulsion comprising: a. water; b. a paraffinic hydrocarbon; and c. a wax component comprising a synthetic olefin wax component, in an amount of about 1 part to about 200 parts, by weight, per 100 parts of said paraffinic hydrocarbon; b) forming the mixture into a structure; and c) drying the structure while permitting hydration of the gypsum to form a gypsum wallboard. The synthetic olefin wax component may be as described hereinabove. In one embodiment, the structure may be an assembly and the method may further comprise placing a layer of the mixture on a first liner, disposing a second liner on the layer in opposed relationship with the first liner to form the assembly of the first and the second liners with the layer sandwiched therebetween. In a further embodiment of the invention, the emulsion further comprises polyvinyl alcohol, which may be present in an amount of about 1 part to about 50 parts, by weight, per 100 parts of said paraffin hydrocarbon.

Also within the scope of the invention is a montan wax substitute for use in an aqueous montan-based wax emulsion comprising a paraffinic hydrocarbon, a wax component, water and polyvinyl alcohol, wherein the wax component comprises the montan wax substitute, and the montan wax substitute comprises a synthetic olefin wax component selected from the group consisting of (i) a synthetic normal α-olefin wax; (ii) a synthetic olefin wax of a carbon chain length of about 20 or more carbon atoms, that is modified by oxidizing and/or by refining through distillation or stripping; and (iii) combinations thereof.

### DETAILED DESCRIPTION OF THE INVENTION

Applicants herein, after significant research for a suitable montan wax substitute have surprisingly found that such as substitute could include a synthetic olefin wax component not previously adopted for use in the building products area, and will work exceptionally well in a wax emulsion for building products to provide good water-resistant properties, better supply capability and reduced cost. Thus, such synthetic olefin wax components can be excellent montan wax substitutes.

Such materials include a synthetic olefin wax component which preferably include synthetic α-olefin waxes, such as normal α-olefin waxes and/or synthetic olefin waxes as described herein. Useful materials within this general category are supplied and available from for example, Chevron-Phillips Chemical Company LP, The Woodlands, Tex. under the name Modified 30+ HA Wax (CAS No. 1003863-31-7; product Numbers 0001103509 and 0001103513). Other preferred materials for such wax emulsions for building products, such as water-resistant gypsum wallboard are described as preferred pour point depressants for hydrocarbon formulations such as oils in U.S. Publication No. 2007/0095723 A1 of Chevron. This publication describes several types of possible pour point depressants, and synthetic olefin waxes of note for use herein are those that are formed as olefin streams from ethylene oligomerization, from cracking of heavy waxes (Fischer Tropsch waxes) and mixtures of paraffins and olefins, as well as normal α-olefin waxes and oxidized waxes.

Most preferred are the synthetic olefin waxes, and more particularly those which are formed of a mixture of: olefins having a chain length of about 28 to about 54 carbons; one or more of the following materials in either or both of unsaturated and saturated forms: aldehydes, ketones, carboxylic acids and esters; and dimers prepared from olefins having carbon chain lengths of about 28 to about 54 so as to have dimer carbon chain lengths of about 65 to about 108.

The disclosure of 2007/0095723 describing such pour point depressant synthetic olefin waxes, normal α-olefin waxes, and oxidized waxes is incorporated in relevant part herein by reference. Derivatives of Chevron α-olefins with carbon numbers above 20 are designated by Chevron for use as pour point depressants. Chevron also notes that such wax fractions are also able to be chemically modified. Preferred amongst Chevron's class of synthetic olefins are α-olefins of carbon chain lengths of at least about 20, more preferred are those about 26 to about 28 and higher, and most preferred are those of 30+ chain length, which may be used as pour point depressants in the art, and α-olefin synthetic materials of about 26 carbons or more, including such materials after chemical modification. All of such materials described hereinabove are within the scope of the “synthetic olefin wax component” as that term is used herein.

The synthetic normal α-olefin waxes and synthetic olefin waxes used in the synthetic olefin wax component herein, alone or in various combinations thereof, preferably are of carbon chain lengths of at least about 20, more preferably at least about 26 and most preferably at least about 30 or more carbon atoms, are also preferably modified either by oxidizing and/or narrowing the molecular weight distribution to refine the wax by various techniques known in the art or to be developed such as various stripping techniques, distillation techniques and the like, and more preferably oxidizing and refining. Even more preferably they may be synthetic olefin waxes formed from mixtures of olefins, saturated and unsaturated ketones, aldehydes, carboxylic acids and/or esters, and olefinic dimers, as noted above.

Preferred materials having such compounds are included within wax emulsions of the same or similar nature to those already used in the building materials art that are based on montan wax and these materials may be used as functional substitutes for montan waxes or for other montan wax substitutes.

In preparing aqueous emulsions using the formulations herein, the aqueous emulsions of the invention preferably comprise a paraffinic hydrocarbon, a synthetic olefin wax component, and water. Other additives may be provided, such as those conventionally employed in emulsions for different purposes including emulsifiers to assist in formation of the emulsion, including stabilizers, such as polyvinyl alcohol (which is preferably hydrolyzed at least 98%), and other useful materials that are known or to be developed to assist in stabilization of the emulsion, rheological agents, thickeners, compatibilizers, colorants, fillers, preservatives, saponifying agents, dispersants, surfactants and the like.

The paraffinic wax may be any suitable paraffin-based wax that functions compatibly with the synthetic olefin wax and the resulting wax emulsion, and is preferably one having a melting point of about 40° C. to about 80° C., which properties are favorable for water-resistant wallboard manufacture, although for other building products applications such as for oriented strand board, other paraffin waxes may be used as well.

In preparing the emulsion herein, while it is based on a montan wax substitute, the wax component of the emulsion may include optional montan wax, or another suitable montan wax substitutes such as those mentioned elsewhere herein in the Examples, including natural carnauba wax, palm wax, Fischer-Tropsch wax, polyethylene wax, oxidized polyethylene wax, polymeric alkenes and their derivatives, siloxanes (with and without catalytic or other additives, which are known for use as water-resistant wax formulation substitutes for preparing water-resistant gypsum wallboard as described in U.S. Patent Publication No. 2006-0035112-A1 for example), bleached or refined montan and synthetic carnauba wax and the like, in a blend with the synthetic olefin wax component, wherein such blends may be from about 99:1 to about 1:99 synthetic olefin wax component to another substitute montan wax or montan wax, more preferably about 80:20 to about 20:80, still more preferably about 70:30 to about 30:70, and most preferably 60:40 to 40:60, provided the formulation is not 100% montan. The synthetic olefin wax component (or blend of synthetic olefin wax with montan wax or another montan wax substitute) is preferably included in the formulation in a total amount of about 1 part to about 200 parts, preferably about 1 part to about 50 parts, by weight, per 100 parts of the paraffinic hydrocarbon.

In preferred embodiments, a stabilizer is provided to the emulsion. Preferably, the stabilizer is polyvinyl alcohol or a similar material, and preferably a polyvinyl alcohol which is prepared by hydrolysis of polyvinyl acetate and is preferably a substantially completely or fully hydrolyzed polyvinyl alcohol. Most preferably it is at least about 90% hydrolyzed polyvinyl alcohol, and more preferably 97% to 100% hydrolyzed polyvinyl alcohol. Most preferably the polyvinyl alcohols used are soluble in water at elevated temperatures of about 60° C. to about 95° C., but are insoluble in cold water. The polyvinyl alcohol may be present in an amount of about 1 part to about 50, preferably about 1 part to about 20 parts, by weight, per 100 parts of the paraffinic wax. The polyvinyl alcohol can enhance water resistance.

The water used to prepare the aqueous emulsion is generally used in an amount of about 35% to about 80%, preferably about 50% to about 65%, by weight, of the emulsion.

Suitable emulsifiers for use in the emulsion of the invention include nonionic surfactants such as alkylphenoxypoly(ethyleneoxy)ethanols, sorbitan fatty acid esters and polyoxyethylene sorbitan fatty acid esters and anionic surfactants such as saponified fatty acids, and, if used, may be present in an amount of about 0.1% to about 5%, by weight, of the emulsion. Other generally known emulsifiers or those to be developed which are useful in wax emulsions and which do not have a deleterious effect on the formulation may be used.

Suitable saponifying agents for use in the emulsion of the invention include alkali metals, preferably potassium hydroxide, ammonium hydroxide, magnesium sulfate, sodium hydroxide or a similar material, and most preferably potassium hydroxide. Saponifiers may be present in an amount of no greater than about 5 weight percent of the emulsion, preferably no greater than about 2 weight percent and most preferably about 0.01 weight percent to about 1 weight percent. Other saponifying agents known or to be developed which are known to be useful in wax emulsions may be used as well.

Dispersants or surfactants of types known in the art may be used. Preferred dispersants, include, but are not limited to those having a sulfur or a sulfur-containing group(s) in the compound such as sulfonic acids (R—S(═O)₂—OH) and their salts, wherein the R groups may be otherwise functionalized with hydroxyl, carboxyl or other useful bonding groups. Preferred are higher molecular weight sulfonic acid compounds such as lignosulfonic acid, naphthalene sulfonic acid, the sulfonate salts of these acids and derivatized or functionalized versions of these materials. In addition, other dispersants known in the art for use in wax emulsions, such as magnesium sulfate; ammonium hepta molybdate/starch combinations; non-ionic surfactants, ionic surfactants, zwitterionic surfactants and mixtures thereof; and alkyl quaternary ammonium montmorillonite clay as well as other known dispersants may be used. Similar materials may also be used herein, provided they are compatible with and perform well with the formulation components.

Dispersants and/or surfactants are preferably present in an amount of about 0.01 percent by weight to about 2 percent by weight of the wax emulsion, and preferably about 0.1 percent to about 2 percent by weight of the wax emulsion

In one method of manufacture of the aqueous emulsion, the paraffinic hydrocarbon and the synthetic olefin wax component (or other blended waxes) are each heated to the molten state and are then blended together. A hot aqueous solution of the polyvinyl alcohol containing the emulsifiers, stabilizers and other components may then be passed with the hot blend of the waxes through a colloid mill and the resulting emulsion is allowed to cool.

Alternatively, a homogenizer may be used instead of a colloid mill. Such homogenizers may be the same general type of equipment used to homogenize milk and other products. In such a method, a mixture of the wax component and the emulsifying components are fed under high pressure (typically about 1500 psi to about 3500 psi) to emulsify the waxes and create a smaller particle size than is typically associated with use of a colloid mill. It will be understood to one skilled in the art based upon this disclosure that other manufacturing methods and types of equipment and procedures for preparing the emulsion can be used, as are known or which may be developed in the art. The emulsion of the invention may also readily be reformed by agitation, in the event that emulsified components of the emulsion separate on storage.

In preparing a gypsum wallboard using this emulsion, an aqueous slurry of the gypsum material is prepared. The aqueous emulsion of the invention is added to the slurry and mixed with the slurry in proportions to provide about 0.5 parts by weight to about 20 parts by weight of the emulsion solids per 100 parts of gypsum. Such compositions may be varied in accordance with conventional gypsum formulation requirements in the art of gypsum manufacture. Other ingredients such as foaming agents, dispersants and set accelerators may be included in the slurry.

In preparing wallboard from such a settable gypsum formulation, the mixture of gypsum slurry and emulsions of the invention can be applied to a first sheet of wallboard liner to form a layer of the gypsum mixture thereon. A second sheet of liner may then be disposed on top of the deposited layer to form a structure in the manner of a wallboard assembly or in which the first and second sheets are in opposed, facing relationship and have the layer of the gypsum mixture therebetween. Alternatively, the gypsum slurry may be prepared directly into a liner-less wallboard structure using manufacturing methods involving press-in-place molding and similar techniques, such that reference to gypsum wallboard herein, is not restricted to liner-covered wallboard. However, it should be understood that any manufacturing technique for making wallboard including a settable gypsum founulation is within the scope of the invention described herein, such as for example, wallboard manufactured with glass mats on the exterior surfaces instead of standard liners.

The resulting structure or assembly may then be dried, such as by oven drying to remove excess water not needed for hydration of the gypsum, to leave finished gypsum wallboard. If liners are used, they may be formed of paper or may comprise fiberglass or organic fiber mats as well. The application will now be described with reference to the following non-limiting examples.

### EXAMPLES

A comparative study was conducted among various possible substitutes for montan wax and using a standard commercial montan wax formulation as in Table 1. In Table 2, various Inventive Examples 1-4 are shown in comparison with a Montan wax control having the results as shown. In Table 3, other comparative potential substitutes for montan wax and accompanying results are shown.

TABLE 1

| Component | Percentage |
|---|---|
| Water | 60.4% |
| Potassium hydroxide | 0.4% |
| Polyvinyl alcohol (98% + hydrolyzed) | 2.4% |
| Lignosulfonate dispersant | 1.5% |
| Paraffin wax (melting point between 130° F. | 33.2% |
| and 150° F. | |
| Montan wax (unrefined Romonta GmbH | 2.1% |
| regular wax) | |

In these Examples, various criteria and properties were evaluated, specifically % water absorption (which was measured using a two-hour soak test at 71° F.), slurry viscosity (measured in seconds using a Number 4 ford cup), formulation viscosity (standard Brookfield viscosity), pH and particle size (measured using an analyzer and taking the volumetric mean). Percentage solids for the formulations were kept in a standard target range of about 38 to about 42 percent by weight. In addition, emulsion and foaming stability were observed for the various samples. Samples were evaluated as potential or good substitutes for montan, and with respect to criteria evaluated for use in water-resistant gypsum wallboard, with water absorption was viewed as a significant criteria (preferred absorption percentages being no greater than about 6% water absorption, and most preferred being no greater than about 5%) as well as with respect to the criteria of cost availability and other industrial use factors.

After sample evaluations of various synthetic olefin waxes obtained from Chevron Phillips, several preferred samples were selected for use in the inventive Examples herein.

Among the preferred materials were Chevron Phillips H1413-86-5 (used in Inventive Examples 1 and 2) and Chevron Phillips H1413-91A (used in Inventive Example 3). Those Examples are shown below in Table 2:

TABLE 2

| Properties/ | Inventive | Inventive | Inventive | Control |
|---|---|---|---|---|
| Criteria | Example 1 | Example 2 | Example 3 | Aqualite ® 70 |
| Percentage | 38.7 | 40.0 | 39.0 | 39.2 |
| Solids (%) | | | | |
| Formulation | 140 | 140 | 160 | 120 |
| Viscosity | | | | |
| (cps) | | | | |
| pH | 12.0 | 11.5 | 11.6 | 11.8 |
| Particle Size | 4.2 | 3.7 | 2.9 | 3.5 |
| (μ) | | | | |
| Water | 5.62/4.62 | 4.8/5.3 | 5.2/5.5 | 4.9 |
| Absorption | | | | |
| (%) | | | | |
| Slurry | 13.3 | 15.1 | 17.1 | 16.8 |
| Viscosity | | | | |
| (sec) | | | | |
| Sample Patty | 16.5 | 17.5 | 15.00 | 17.0 |
| Size (cm) | | | | |
| Emulsion | Good | Good | Good | Good |
| Stability | | | | |
| Foaming | Good | Good | Slight Foam | Good |
| Stability | | | | |

To evaluate potential montan substitutes prior to finding the inventive substitute noted in Table 2 and described herein, applicants also did significant testing of other potential montan substitutes, including carnauba wax (Carnauba T-3 and T-4), Fischer Tropsch wax from Sasol (A1 and A28), refined montan waxes (bleached waxes) (Clariant® EMS, ESL and S waxes), palm wax (HP5401-C, HP5601-A) and oxidized palm wax (HPX OX-50) in comparison with control formulations. Most of the potential comparative substitutes worked reasonably well in combination with montan wax, but that did not resolve the need to completely replace montan. Some of these materials proved inadequate in performance (palm wax). Further, while some performed well with respect to water absorption, they were found to be cost prohibitive substitutes (carnauba wax) and/or in insufficient supply or difficult to obtain (bleached montan—while good is still refined from montan) such that they were not viable substitutes from a manufacturing perspective. Similarly, while Sasol Fischer Tropsch waxes seemed to perform well, they were of limited availability and difficult to process. Examples of comparative testing of potential montan substitutes are shown below in Table 3:

TABLE 3

| | | | | | Water | Patty |
|---|---|---|---|---|---|---|
| | | Solids | Formulation | | Absorption | Size |
| Sample | Montan Substitute Component | (%) | Viscosity (cps) | pH | (%) | (cm) |
| Control 1 | Control (Montan) | 38.7 | 148 | 11.4 | 4.8 | 16.5 |
| Control 2 | Control (Montan) | 38.3 | 372 | 11.8 | 5.5 | 17.0 |
| Inventive 1 | Chevron Phillips H1413-86-5 (synthetic olefin) | 38.7 | 140 | 12.0 | 5.62/4.62 | 16.5 |
| Comp. 1 | Carnauba T-4 (carnauba wax) | 38.3 | 336 | 12.1 | 5.6 | 17.5 |
| Comp. 2 | Koster Keunen K83H (synthetic carnauba) | — | — | — | — | — |
| Comp. 3 | Baker Petrolite Ceramer 1608 (polymeric alkene) | 39.2 | 128 | 8.2 | 9.1 | 15.00 |
| Comp. 4 | Baker Petrolite CA-11 (reaction product - oxidized | 39.2 | 57 | 9.4 | 8.6 | 15.5 |
| | alkenes) | | | | | |
| Comp. 5 | HP-5401-C (Palm wax) | 38.3 | 240 | 7.7 | 22.6 | — |
| Comp. 6 | HP 5601-A (Palm wax) | 39.0 | 272 | 8.2 | 8.8 | — |
| Comp. 7 | HP Synthetic Montan A (Palm Wax) | 39.3 | 296 | 11.4 | 49.2 | 16.25 |
| Comp. 8 | HP Synthetic Montan C (Palm Wax) | 39.5 | 312 | 11.5 | 64.4 | 17.00 |
| Comp. 9 | Sasolwax A1 (Fischer-Tropsch) | 39.3 | 80 | 11.7 | 6.8 | 14.0 |
| Comp. 10 | Sasolwax A28 (Fischer-Tropsch) | 40.1 | 132 | 12.1 | 6.6 | 17.0 |
| Comp. 11 | Honeywell AC-629A (oxidized polyethylene wax) | 39.5 | 2080 | 11.2 | 8.4 | 14.75 |
| Comp. 12 | Baker Petrolite Cardis 36 (oxidized polyethylene wax) | 39.2 | 184 | 11.3 | 9.7 | 15.25 |
| Comp. 13 | Baker Petrolite Cardis 320 (oxidized polyethylene wax) | 39.2 | 180 | 11.3 | 6.4 | 16.0 |

With regard to the wax emulsions made with the inventive substitute montan wax in the form of a synthetic olefin wax, such formulations perform equivalently or better than the control samples and present an inexpensive, easy to process wax formulation which serves as an excellent wax emulsion based on a unique montan wax substitute.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. An aqueous wax emulsion, comprising:
water;
a paraffinic hydrocarbon; and
a wax component comprising a synthetic olefin wax component, wherein the synthetic olefin wax component is selected from the group consisting of (i) a synthetic normal α-olefin wax; (ii) a synthetic olefin wax of a carbon chain length of about 20 or more carbon atoms, that is modified by oxidizing and/or by refining through distillation or stripping; and (iii) combinations thereof.

2. The aqueous wax emulsion according to claim 1, wherein the synthetic olefin wax component is the synthetic olefin wax (ii) and has a carbon chain length of at least 28.

3. The aqueous wax emulsion according to claim 2, wherein the synthetic olefin wax component is the synthetic olefin wax (ii) and has a carbon chain length of at least 30.

4. The aqueous wax emulsion according to claim 1, wherein the synthetic olefin wax component is the synthetic olefin wax (ii) and comprises a mixture of one or more of (a) an olefin having a carbon chain of about 28 to about 54 carbons; (b) one or more component selected from an aldehyde, a ketone, a carboxylic acid and a carboxylic ester; and (c) a dimer prepared from olefins having carbon chain lengths of about 28 to about 54 so as to have dimer carbon chain lengths of about 65 to about 108.

5. The aqueous wax emulsion according to claim 1, wherein the wax component further comprises montan wax in a blend with the synthetic olefin wax component.

6. The aqueous wax emulsion according to claim 1, wherein the wax component is a blend of the synthetic olefin wax component with one or more of natural or synthetic carnauba wax, palm wax, Fischer-Tropsch wax, a polymeric alkene, and an oxidized polyethylene wax.

7. The aqueous wax emulsion according to claim 1 wherein the synthetic olefin wax component is from about 1 percent to about 100 percent of the wax component.

8. The aqueous wax emulsion according to claim 7, wherein the synthetic olefin wax component is from about 20 percent to about 80 percent of the wax component.

9. The aqueous wax emulsion according to claim 8, wherein the synthetic olefin wax component is from about 30 percent to about 70 percent of the wax component.

10. The aqueous wax emulsion according to claim 9, wherein the synthetic olefin wax component is from about 40 percent to about 60 percent of the wax component.

11. The aqueous wax emulsion according to claim 1, further comprising a stabilizer.

12. The aqueous wax emulsion according to claim 11, wherein the stabilizer is polyvinyl alcohol.

13. The aqueous wax emulsion according to claim 12, wherein the polyvinyl alcohol is present in an amount of about 1 part to about 20 parts, by weight, per 100 parts of said paraffin hydrocarbon.

14. The aqueous wax emulsion according to claim 13, wherein the polyvinyl alcohol is about 97% to about 100% hydrolyzed polyvinyl alcohol.

15. The aqueous wax emulsion according to claim 1, further comprising a saponifying agent.

16. The aqueous wax emulsion according to claim 15, wherein the saponifying agent is an alkali metal.

17. The aqueous wax emulsion according to claim 16, wherein the alkali metal is potassium hydroxide.

18. The aqueous wax emulsion according to claim 1, further comprising at least one of a dispersant and a surfactant.

19. The aqueous wax emulsion according to claim 18, wherein the dispersant comprises sulfur or a sulfur-containing group.

20. The aqueous wax emulsion according to claim 19, wherein the dispersant is lignosulfonate.

21. The aqueous wax emulsion according to claim 1, wherein the paraffinic hydrocarbon is a paraffin wax having a melting point of about 40° C. to about 80° C.

22. A settable gypsum composition suitable for forming a water-resistant gypsum product comprising:
a) 100 parts by weight of gypsum; and
b) about 0.5 part to about 20 parts, by weight, of emulsion solids, per 100 parts, by weight, of gypsum, of an aqueous emulsion comprising:
i) water;
ii) a paraffinic hydrocarbon; and
iii) a wax component comprising synthetic olefin wax component, wherein the wax component is present in an amount of about 1 part to about 200 parts, by weight, per 100 parts of the paraffinic hydrocarbon.

23. The settable gypsum composition according to claim 22, wherein the synthetic olefin wax component is selected from the group consisting of (i) a synthetic normal α-olefin wax; (ii) a synthetic olefin wax of a carbon chain length of about 20 or more carbon atoms, that is modified by oxidizing and/or by refining through distillation or stripping; and (iii) combinations thereof.

24. The settable gypsum composition according to claim 23, wherein the synthetic olefin wax component is the synthetic olefin wax (ii) and has a carbon chain length of at least 30.

25. The composition according to claim 22, further comprising a stabilizer.

26. The composition according to claim 25, wherein the stabilizer is polyvinyl alcohol in an amount of about 1 part to about 50 parts, by weight, per 100 parts of said paraffin hydrocarbon.

27. The composition according to claim 26, wherein the polyvinyl alcohol is about 97% to about 100% hydrolyzed polyvinyl alcohol.

28. A water-resistant gypsum board having a core sandwiched between a pair of liners, said core comprising a set composition of claim 22.

29. A method of manufacturing a water-resistant gypsum board comprising:
a) forming a mixture of
i) 100 parts by weight of gypsum; and
ii) about 0.5 part to about 20 parts, by weight, of emulsion solids, per 100 parts, by weight, of the gypsum, of an aqueous emulsion comprising:
a. water;
b. a paraffinic hydrocarbon; and
c. a wax component comprising a synthetic olefin wax component in an amount of about 1 part to about 200 parts, by weight, per 100 parts of said paraffinic hydrocarbon;
b) forming the mixture into a structure; and
c) drying the structure while permitting hydration of the gypsum to form a gypsum wallboard.

30. The method according to claim 29, wherein the synthetic olefin wax component in the mixture is selected from the group consisting of (i) a synthetic normal α-olefin wax; (ii) a synthetic olefin wax of a carbon chain length of about 20 or more carbon atoms, that is modified by oxidizing and/or by refining through distillation or stripping; and (iii) combinations thereof.

31. The method according to claim 29, wherein the structure is an assembly and the method further comprising placing a layer of the mixture on a first liner, disposing a second liner on the layer in opposed relationship with the first liner to form the assembly of the first and the second liners with the layer sandwiched therebetween.

32. The method according to claim 29, wherein the mixture further comprises polyvinyl alcohol in an amount of about 1 part to about 50 parts, by weight, per 100 parts of said paraffin hydro carbon.

33. A montan wax substitute for use in an aqueous montan-based wax emulsion comprising a paraffinic hydrocarbon, a wax component, water and polyvinyl alcohol, wherein the wax component comprises the montan wax substitute in an amount of about 1 part to about 100 percent of the wax component in the emulsion, and the montan wax substitute comprises a synthetic olefin wax component selected from the group consisting of (i) a synthetic normal α-olefin wax; (ii) a synthetic olefin wax of a carbon chain length of about 20 or more carbon atoms, that is modified by oxidizing and/or by refining through distillation or stripping; and (iii) combinations thereof.
